(19) United States
(12) Patent Application Publication
(10) Pub. No.: US 2005/0201964 A1
(43) Pub. Date: Sep. 15, 2005

(54) Composition to permanently reshape the hair containing at least one dicarboxydithiol

(76) Inventors: Gerard Malle, Villiers-Sur-Morin (FR); Christian Blaise, Saint-Mande (FR)

Correspondence Address: Thomas L. Irving;FINNEGAN, HENDERSON, FARABOW, GARRETT & DUNNER , L.L.P., 901 New York Avenue, N.W., Washington, DC, 20001-4413, US

(21) Appl. No.: 11/068,851

(22) Filed: Mar. 2, 2005

Related U.S. Application Data
(60) Provisional application No. 60/559,004, filed on Apr. 5, 2004.

(30) Foreign Application Priority Data
Mar. 2, 2004 (FR) 04 50417

(51) Int. Cl. A61K 7/09

(52) U.S. Cl. 424/70.2; 424/70.5

(57) ABSTRACT

The present invention relates to an aqueous reducing composition, for the first step of a process to permanently reshape the hair, comprising in a cosmetically acceptable medium, as reducing agents, one or more dicarboxydithiols of general formula (I) and/or of general formula (II): 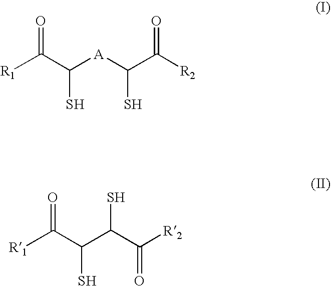 wherein:
R₁, R₂, R′₁, R′₂ independently represent OR₃ or NR₄R₅, where R₃ represents H or a linear or branched C₁-C₅ alkyl group optionally substituted by one or two OR₇ radicals, where R₇ represents H or a C₁-C₃ alkoxy radical,
R₄, R₅ are independently H or a linear or branched C₁-C₅ alkyl group optionally substituted by one or two radicals selected from hydroxy, methoxy, ethoxy, methylamino, ethylamino and dimethylamino radicals,
R₁ and R₂ not being simultaneously a hydroxyl radical and R′₁ and R′₂ not being simultaneously a hydroxyl radical;
A either represents (CH₂)ₙ where n is an integer ranging from 1 to 4, or a —(CH₂)ₘ—X—(CH₂)ₚ— radical, where m is 1 or 2, p is 1 or 2 and X represents O, S, NHCO or NR₆, where R₆ represents H or CH₃, and/or one or more organic and mineral salts thereof.

This application claims benefit of U.S. Provisional Application No. 60/559,004, filed Apr. 5, 2004.

The present invention relates to a reducing cosmetic composition for the first step of a process to permanently reshape the hair, comprising at least one dicarboxydithiol as a reducing agent. It also relates to a method for permanently reshaping the hair, which uses these dicarboxydithiols.

The most usual art to obtain a permanent reshaping of the hair consists, in a first step, in opening the keratin disulfide bonds (cystine) with a composition comprising a reducing agent, and preferably after rinsing the hair, in reforming in a second step said disulfide bonds by applying to the hair which has been placed beforehand under tension with curlers or equivalent, or set in form or straightened by other means, an oxidizing composition also called fixing solution, so as to give the desired form to the hair. This method enables indifferently either the waving of the hair, or the decurling, the backcombing or the straightening thereof.

Reducing-compositions usually used during the first step of a permanent waving operation comprise sulfites, bisulfites, and preferably thiols, as reducing agents. Among these latest, those traditionally used are thioglycolic acid, cysteamine, thiolactic acid, cysteine and glycerol monothioglycolate. Thioglycolic acid is particularly effective to reduce keratin disulfide bonds at alkaline pH, especially as ammonium thioglycolate, and represents the product which is the most often used in permanent waving. Nevertheless, it has been noted that thioglycolic acid had to be used in sufficiently basic medium (practically at pH 8.5 or above) to obtain a strong enough curling. As a drawback, the thioglycolic acid-alkaline pH combination, not only has an unpleasant odor, which has to be overcame by the use of fragrances that are more or less effective to mask bad odors, but causes damage to the hair fibre as well.

Sulfites and bisulfites have been used prior to thiols in general and to thioglycolic acid in particular. Unlike thiols, they are used at acidic pH, generally between 4 and 6. Nevertheless, the intensity of curling obtained is much lower and is still far from being satisfactory.

Cysteine produces a much weaker odor than thioglycolic acid does, but the curling degree obtained is also much lower and very unsatisfactory. Moreover, cysteine has to be used at very alkaline pH.

Glycerol monothioglycolate also has a very unpleasant odor. By contrast, it is used at pH close to neutrality, but its performance is appreciably lower than thioglycolic acid.

Cysteamine may be used on a broader pH range. It is almost as much effective as thioglycolic acid, but causes also the hair fibre to degrade significantly.

Many different studies have been conducted to compensate for the drawbacks of these reducing agents and it has been proposed to that end to use new reducing compounds or systems. Nevertheless, very few dithiols have been proposed. Two dithiols had been very intensively studied in the past i.e. dithiothreitol DTT and 2,5-dimercaptoadipic acid, but they have never been developed to permanently reshape the hair, especially because of DTT's unbearable odor and because dimercaptoadipic acid was not sufficiently active. More recently, it has already been proposed in the european application EP-A-0721772 to use 2,3-dimercaptosuccinic acid which appeared to be less effective than thioglycolic acid. It has also been suggested in U.S. Pat. No. 5,350,572 to use polyoxyethyleneglycol dimercaptoalkylesters. Although these compounds are relatively effective, they are difficult to preserve in the long term.

After having carried out various studies, the applicant has now quite surprisingly discovered a new category of dicarboxydithiols which enable to produce a reducing composition, which results in an intensive and long lasting hold curling, also with less damage to the hair, as compared to previous compositions.

It is thus one object of the present invention to provide an aqueous reducing composition for the first step of a permanent hair reshaping process, comprising in a cosmetically satisfactory medium, one or more dicarboxydithiols as reducing agents, having the following general formula (I) and/or the following general formula (II): 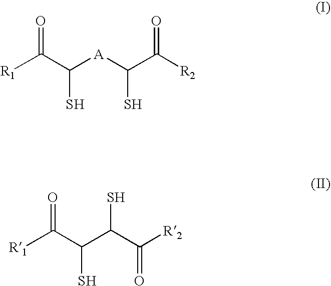
wherein:
R₁, R₂, R′₁, R′₂ independently represent OR₃ or NR₄R₅, where R₃ is H or a linear or branched, C₁-C₅ alkyl group, optionally substituted by one or two OR₇ radicals, where R₇ is H or C₁-C₃ alkoxy radical,
R₄, R₅ are independently H or a linear or branched C₁-C₅alkyl group optionally substituted by one or two radicals selected from hydroxy, methoxy, ethoxy, methylamino, ethylamino and dimethylamino radicals,
R₁ and R₂ not being simultaneously a hydroxyl radical and R′₁ and R′₂ not being simultaneously a hydroxyl radical;
A either represents (CH₂)ₙ, where n is an integer ranging from 1 to 4, or a —(CH₂)ₘ—X—(CH₂)ₚ— radical, where m is 1 or 2, p is 1 or 2 and X represents O, S, NHCO or NR₆, where R₆ represents H or CH₃,
and/or one or more organic or mineral salts thereof.

By “permanent reshaping”, it is intended herein to mean the permanent curling (permanent waving), the uncurling or the straightening of the hair.

Amongst the compounds having the general formula (I) and (II), the preferred compounds are as follows:
2,3-dimercapto-4-methylamino-4-oxo-butanoic acid,
2,3-dimercapto-4-ethylamino-4-oxo-butanoic acid,
2,3-dimercapto-4-propylamino-4-oxo-butanoic acid,
2,3-dimercapto-4-butylamino-4-oxo-butanoic acid,
2,3-dimercapto-4-[(1-methylethyl)amino]-4-oxo-butanoic acid,
2,3-dimercapto-4-[(2-methylpropyl)amino]-4-oxo-butanoic acid,
2,3-dimercapto-4-[(3-methylbutyl)amino]-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-butanedioic acid, monomethylester,
2,3-dimercapto-butanedioic acid, monoethylester,
2,3-dimercapto-butanedioic acid, monopropylester,
2,3-dimercapto-butanedioic acid, mono(1-methylethyl)ester,
2,3-dimercapto-butanedioic acid, monobutylester,
2,3-dimercapto-butanedioic acid, mono(2-methylpropyl)ester,
2,3-dimercapto-butanedioic acid, monopentylester,
2,3-dimercapto-butanedioic acid, mono(3-methylbutyl)ester,
2,3-dimercapto-butanediamide,
2,3-dimercapto-N,N′-dimethyl-butanediamide,
2,3-dimercapto-N,N′-diethyl-butanediamide,
2,3-dimercapto-N,N′-bis(2-hydroxyethyl)-butanediamide,
2,3-dimercapto-N,N′-bis(2-hydroxypropyl)-butanediamide,
2,3-dimercapto-butanedioic acid, dimethylester,
2,3-dimercapto-butanedioic acid, diethylester,
2,3-dimercapto-butanedioic acid, dipropylester,
2,3-dimercapto-butanedioic acid, bis(1-methylethyl)ester,
2,3-dimercapto-butanedioic acid, dibutylester,
2,3-dimercapto-butanedioic acid, bis(2-methylpropyl)ester,
2,3-dimercapto-butanedioic acid, bis(1,1-dimethylethyl)ester,
2,3-dimercapto-butanedioic acid, bis(1-methylpropyl)ester,
2,3-dimercapto-butanedioic acid, bis(3-methylbutyl)ester,
2,3-dimercapto-butanedioic acid, bis(1-ethylpropyl)ester,
2,3-dimercapto-butanedioic acid, bis(2-methoxyethyl)ester,
2,3-dimercapto-butanedioic acid, bis(2-ethoxyethyl)ester,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-butylamino-5-oxo-pentanoic acid,
2,4-dimercapto-5-[(1-methylethyl)amino]-5-oxo-pentanoic acid,
2,4-dimercapto-5-[(2-methylpropyl)amino]-5-oxo-pentanoic acid,
2,4-dimercapto-5-[(3-methylbutyl)amino]-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-pentanedioic acid, monomethylester,
2,4-dimercapto-pentanedioic acid, monoethylester,
2,4-dimercapto-pentanedioic acid, monopropylester,
2,4-dimercapto-pentanedioic acid, mono(1-methylethyl)ester,
2,4-dimercapto-pentanediamide,
2,4-dimercapto-N,N′-dimethyl-pentanediamide,
2,4-dimercapto-N,N′-diethyl-pentanediamide,
2,4-dimercapto-N,N′-bis(2-hydroxyethyl)-pentanediamide,
2,4-dimercapto-N,N′-bis(2-hydroxypropyl)-pentanediamide,
2,4-dimercapto-pentanedioic acid, dimethylester,
2,4-dimercapto-pentanedioic acid, diethylester,
2,4-dimercapto-pentanedioic acid, dipropylester,
2,4-dimercapto-pentanedioic acid, dibutylester,
5-carbamoyl-2,5-dimercapto-valeric acid,
2,5-dimercapto -6-methylamino-6-oxo -hexanoic acid,
2,5-dimercapto -6-ethylamino-6-oxo-hexanoic acid,
2,5-dimercapto-6-propylamino-6-oxo-hexanoic acid,
2,5-dimercapto-6-[(2-hydroxyethyl)amino]-6-oxo-hexanoic acid,
2,5-dimercapto-6-[(2-hydroxypropyl)amino]-6-oxo-hexanoic acid,
2,5-dimercapto-6-[(2-dimethylaminoethyl)amino]-6-oxo-hexanoic acid,
2,5-dimercapto-6-[(3-dimethylaminopropyl)amino]-6-oxo-hexanoic acid,
2,5-dimercapto-hexanedioic acid, monomethylester,
2,5-dimercapto-hexanedioic acid, monoethylester,
2,5-dimercapto-hexanedioic acid, monopropylester,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
2,5-dimercapto-N,N, N′,N′-tetramethyl-hexanediamide,
2,5-dimercapto-hexanedioic acid, dimethylester,
2,5-dimercapto-hexanedioic acid, diethylester,
2,5-dimercapto-hexanedioic acid, dipropylester,
2,6-dimercapto-7-methylamino-7-oxo -heptanedioic acid,
2,6-dimercapto-7-ethylamino-7-oxo-heptanedioic acid,
2,6-dimercapto-7-propylamino-7-oxo-heptanedioic acid,
2,6-dimercapto-7-[(2-hydroxyethyl)amino]-7-oxo-heptanedioic acid,
2,6-dimercapto-7-[(2-hydroxypropyl)amino]-7-oxo-heptanedioic acid,
2,6-dimercapto-7-[(2-dimethylaminoethyl)amino]-7-oxo-heptanedioic acid,
2,6-dimercapto-7-[(3-dimethylaminopropyl)amino]-7-oxo-heptanedioic acid,
2,6-dimercapto-heptanedioic acid, monomethylester,
2,6-dimercapto-heptanedioic acid, monoethylester,
2,6-dimercapto-heptanedioic acid, monopropylester,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
2,6-dimercapto-heptanedioic acid, dimethylester,
2,6-dimercapto-heptanedioic acid, diethylester,
2,6-dimercapto-heptanedioic acid, dipropylester,
2,7-dimercapto-8-methylamino-8-oxo-octanedioic acid,
2,7-dimercapto -8-ethylamino-8-oxo-octanedioic acid,
2,7-dimercapto -8-propylamino-8-oxo-octanedioic acid,
2,7-dimercapto -8-[(2-hydroxyethyl)amino]-8-oxo-octanedioic acid,
2,7-dimercapto-8-[(2-hydroxypropyl)amino]-8-oxo-octanedioic acid,
2,7-dimercapto-8-[(2-dimethylaminoethyl)amino]-8-oxo-octanedioic acid,
2,7-dimercapto-8-[(3-dimethylaminopropyl)amino]-8-oxo-octanedioic acid,
2,8-dimercapto-octanedioic acid, monomethylester,
2,8-dimercapto-octanedioic acid, monoethylester,
2,8-dimercapto-octanedioic acid, monopropylester,
2,8-dimercapto-heptanediamide,
2,8-dimercapto-N,N′-dimethyl-heptanediamide,
2,8-dimercapto-N,N′-bis(2-hydroxyethyl)-heptanediamide,
2,8-dimercapto-N,N′-bis(2-hydroxypropyl)-heptanediamide,
2,8-dimercapto-heptanedioic acid, dimethylester,
2,8-dimercapto-heptanedioic acid, diethylester,
2,8-dimercapto-heptanedioic acid, dipropylester,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-propylamino-5-oxo-pentanoic acid,
2,4-dimercapto-3-oxa-5-butylamino-5-oxo-pentanoic acid,
2,4-dimercapto-3-oxa-5-[(1-methylethyl)amino]-5-oxo-pentanoic acid,
2,4-dimercapto-3-oxa-5-[(2-methylpropyl)amino]-5-oxo-pentanoic acid,
2,4-dimercapto-3-oxa-5-[(3-methylbutyl)amino]-5-oxo-pentanoic acid,
2,4-dimercapto-3-oxa-5-[(2-hydroxyethyl)amino]-5-oxo-pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanedioic acid, monomethylester,
2,4-dimercapto-3-oxa-pentanedioic acid, monoethylester,
2,4-dimercapto-3-oxa-pentanedioic acid, monopropylester,
2,4-dimercapto-3-oxa-pentanedioic acid, mono(1-methylethyl)ester,
2,4-dimercapto-3-oxa-pentanediamide,
2,4-dimercapto-3-oxa-N,N′-dimethyl-pentanediamide,
2,4-dimercapto-3-oxa-N,N′-diethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,4-dimercapto-3-oxa-pentanedioic acid, dimethylester,
2,4-dimercapto-3-oxa-pentanedioic acid, diethylester,
2,4-dimercapto-3-oxa-pentanedioic acid, dipropylester,
2,4-dimercapto-3-oxa-pentanedioic acid, dibutylester,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-propylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-7-[(2-dimethylaminoethyl)amino]-7-oxo-heptanedioic acid,
2,6-dimercapto-4-thia-7-[(3-dimethylaminopropyl)amino]-7-oxo-heptanedioic acid,
2,6-dimercapto-4-thia-heptanedioic acid, monomethylester,
2,6-dimercapto-4-thia-heptanedioic acid, monoethylester,
2,6-dimercapto-4-thia-heptanedioic acid, monopropylester,
2,6-dimercapto-4-thia-heptanediamide,
2,6-dimercapto-4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-4-thia-N,N′-bis(2-hydroxypropyl)-heptanediamide,
2,6-dimercapto-4-thia-heptanedioic acid, dimethylester,
2,6-dimercapto-4-thia-heptanedioic acid, diethylester,
2,6-dimercapto-4-thia-heptanedioic acid, dipropylester,
N-(carboxy-mercapto-methyl)-2-mercapto-malonamic acid,
[(carboxy-mercapto-methyl)-methyl-amino]-mercapto-acetic acid.

The following compounds are especially preferred:
2,3-dimercapto-4-methylamino-4-oxo-butanoic acid,
2,3-dimercapto-4-ethylamino-4-oxo-butanoic acid,
2,3-dimercapto-4-propylamino-4-oxo-butanoic acid,
2,3-dimercapto-4-butylamino-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-butanediamide,
2,3-dimercapto-N,N′-dimethyl-butanediamide,
2,3-dimercapto-N,N′-bis(2-hydroxyethyl)-butanediamide,
2,3-dimercapto-N,N′-bis(2-hydroxypropyl)-butanediamide,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-pentanediamide,
2,4-dimercapto-N,N′-dimethyl-pentanediamide,
2,4-dimercapto-N,N′-bis(2-hydroxyethyl)-pentanediamide,
2,4-dimercapto-N,N′-bis(2-hydroxypropyl)-pentanediamide,
5-carbamoyl-2,5-dimercapto-valeric acid,
2,5-dimercapto-6-methylamino-6-oxo-hexanoic acid,
2,5-dimercapto-6-ethylamino-6-oxo-hexanoic acid,
2,5-dimercapto-6-[(2-hydroxyethyl)amino]-6-oxo-hexanoic acid,
2,5-dimercapto-6-[(2-hydroxypropyl)amino]-6-oxo-hexanoic acid,
2,5-dimercapto-6-[(2-dimethylaminoethyl)amino]-6-oxo-hexanoic acid,
2,5-dimercapto-6-[(3-dimethylaminopropyl)amino]-6-oxo-hexanoic acid,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
2,6-dimercapto-7-methylamino-7-oxo-heptanedioic acid,
2,6-dimercapto-7-ethylamino-7-oxo-heptanedioic acid,
2,6-dimercapto-7-[(2-hydroxyethyl)amino]-7-oxo-heptanedioic acid,
2,6-dimercapto-7-[(2-hydroxypropyl)amino]-7-oxo-heptanedioic acid,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
2,7-dimercapto-8-methylamino-8-oxo-octanedioic acid,
2,7-dimercapto-8-ethylamino-8-oxo-octanedioic acid,
2,7-dimercapto-8-[(2-hydroxyethyl)amino]-8-oxo-octanedioic acid,
2,7-dimercapto-8-[(2-hydroxypropyl)amino]-8-oxo-octanedioic acid,
2,8-dimercapto-heptanediamide,
2,8-dimercapto-N,N′-dimethyl-heptanediamide,
2,8-dimercapto-N,N′-bis(2-hydroxyethyl)-heptanediamide,
2,8-dimercapto-N,N′-bis(2-hydroxypropyl)-heptanediamide,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-[(2-hydroxyethyl)amino]-5-oxo-pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanediamide,
2,4-dimercapto-3-oxa-N,N′-dimethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-heptanediamide,
2,6-dimercapto-4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-4-thia-N,N′-bis(2-hydroxypropyl)-heptanediamide.

In the reducing composition according to the present invention, the reducing agent(s) having the general formula (I) and/or (II), and/or organic and mineral salts thereof, are generally comprised in a quantity between 0.05 and 30%, preferably between 1 and 20% by weight, as compared to the total weight of the reducing composition.

The pH of the composition according to the present invention is preferably between 4 and 11, and more particularly between 6 and 10.

The pH is generally balanced by means of an alkaline agent such as, for example, ammonia, monoethanolamine, diethanolamine, triethanolamine, 1,3-propanediamine, alkaline or ammonium carbonate or bicarbonate, organic carbonate such as guanidine carbonate, alkaline hydroxide or by means of an acidifying agent such as, for example, hydrochloric acid, acetic acid, lactic acid, oxalic acid or boric acid, or by means of ordinary buffers, such as, for example, borate, phosphate or TRIS-buffers (tris(hydroxymethyl)amino-methane-based buffers).

The reducing composition according to the present invention may also comprise one or more other known reducing agents, such as, for example, thioglycolic acid or thiolactic acid and their esters and amides derivatives, especially glycerol monothioglycolate, cysteamine and its acylated (C₁-C₄)-derivatives, such as N-acetyl-cysteamine or N-propionyl-cysteamine, cysteine, N-acetyl-cysteine, thiomalic acid, pantheteine, 2,3-dimercaptosuccinic acid, alkali metal or alkaline-earth metal sulfites or bisulfites, N-(mercaptoalkyl)-ω-hydroxyalkylamides such as those described in the patent application EP-A-354 835, N-mono or N,N-dialkylmercapto-4-butyramides such as those described in the patent application EP-A-368 763, aminomercaptoalkylamides, such as those described in the patent application EP-A-432 000, N-(mercaptoalkyl)succinamic acids and N-(mercaptoalkyl)succinimide derivatives such as those described in the patent application EP-A-465 342, alkylaminomercaptoalkylamides such as those described in the patent application EP-A-514 282, an azeotropic mixture of 2-hydroxypropyl thioglycolate and (2-hydroxy-1-methyl)ethyl thioglycolate described in the patent application FR-A-2 679 448, mercaptoalkylaminoamides such as those described in the patent application FR-A-2 692 481, N-mercaptoalkylalkanediamides described in the patent application EP-A-653 202, as well as formamidine sulfinic acid derivatives such as those described in the application PCT/US01/43124, filed by the present applicant.

In a preferred embodiment, the reducing composition according to the present invention also comprise at least one surfactant of non ionic, anionic, cationic or amphoteric type. Possible examples thereof are the alkyl sulfates, alkyl benzene sulfates, alkyl ether sulfates, alkyl sulfonates, quaternary ammonium salts, alkyl betaines, oxyethylene alkyl phenols, fatty acid alkanolamides, oxyethylene fatty acid esters, as well as other non ionic hydroxypropylether surfactants.

When the reducing composition according to the present invention comprises one or more surfactants, they generally represent at most 30% by weight, and preferably from 0,5 to 10% by weight, as compared to the total weight of the reducing composition.

In order to improve the cosmetic properties of the hair or to reduce or prevent their damaging, the reducing composition may also comprise a treating agent of cationic, anionic, non ionic or amphoteric nature.

Amongst the treating agents that are particularly preferred, there are notably those described in the French patents FR 2 598 613 and FR 2 470 596. Volatile or non volatile, linear or cyclic silicones may also be used as treating agents, as well as mixtures thereof, polydimethylsiloxanes, quaternized polyorganosiloxanes such as those described in the French patent application FR 2 535 730, polyorganosiloxanes with aminoalkyl moieties modified by alkoxycarbonylalkyl moieties, such as those described in the U.S. Pat. No. 4,749,732, polyorganosiloxanes such as polydimethylsiloxane-polyoxyalkyl copolymer i.e. dimethicone copolyol, polydimethylsiloxane with stearoxy end groups (stearoxy-dimethicone), polydimethylsiloxane and dialkylammonium acetate copolymer or polydimethylsiloxane and polyalkyl betaine copolymer described in French patent No 2 197 352, polysiloxanes organo-modified by mercapto or mercaptoalkyl groups, such as those described in the French patent FR 1 530 369 and in the European patent application EP 295 780, as well as silanes such as stearoxytrimethylsilane.

The reducing composition according to the present invention may also comprise one or more other treating ingredients, such as cationic polymers such as those used in the compositions of the French patents no 79.32078 (FR 2 472 382) and 80.26421 (FR 2 495 931), orcationic polymers such as of the ionene type used in the compositions of the Luxemburger patent no 83703, basic aminoacids such as lysine or arginine, acidic aminoacids, such as glutamic acid or aspartic acid, peptides and their derivatives, protein hydrolyzates, waxes, swelling agents and penetrating agents, or agents that are able to increase the reductor's efficiency such as a SiO₂/PDMS mixture (polydimethylsiloxane), dimethylisosorbitol, urea and its derivatives, pyrrolidone, N-alkyl-pyrrolidones, thiamorpholinone, alkyleneglycol or dialkyleneglycol alkylethers such as, for example, propyleneglycol monomethylether, dipropyleneglycol monomethylether, ethyleneglycol monoethylether and diethyleneglycol monoethylether, C₃-C₆ alkanediols such as, for example, 1,2-propanediol, 1,3-propanediol and 1,2-butanediol, 2-imidazolidinone, as well as other compounds such as fatty alcohols, lanoline derivatives, active agents such as panthothenic acid, agents for combating hair loss, anti-dandruff agents, thickeners, suspension agents, sequestering agents, opacifying agents, dyes, sunscreen agents as well as fragrances and preserving agents.

Dicarboxydithiol(s) of the formula (I) and/or (II), and/or organic and mineral salts thereof, have to be placed in a cosmetically acceptable medium.

The reducing composition according to the present invention may thus comprise a solvent such as, for example, ethanol, propanol or isopropanol or glycerol, with a maximal concentration of 20% by weight, as compared to the total weight of the composition.

The vehicle for the compositions according to the present invention is preferably water or a hydroalcoholic solution made of a C₁-C₆ lower alcohol such as ethanol, isopropanol or butanol.

Preferably, the reducing composition according to the present invention is in substantially aqueous form, i.e. it comes as a lotion thickened or not, as a cream thickened or not, or as a gel.

When the compositions are to be used for a hair uncurling or straightening process, the reducing composition comes preferably as a thickened cream, so as to maintain the hair as straight as possible. These creams are made in the form of “heavy” emulsions, for example with a base consisted of glyceryl stearate, glycol stearate, auto-emulsifiable waxes or fatty alcohols.

Liquids or gels containing certain thickeners may also be used, such as carboxyvinyl polymers or copolymers, which “stick” and fix the hair in the smooth position during the exposure time.

The reducing composition according to the present invention can be an exothermic type composition as well, that's to say it causes a certain heating-up when applied onto the hair, which is pleasant for the user who is experiencing the permanent waving or the uncurling first step.

Some dicarboxydithiols having the general formula (I) or (II) are new compounds and their method of preparation will be described thereinafter in more detail. The known compounds are generaly prepared according to the procedures described in the following references:
Polyhedron 1999, 18 (25), 3257-3262,
Chemical Research in Toxicology 1996, 9(6), 965-969,
Forschungszentrum Rossendorf e.V.; 1996, 102-104,
Drug and Chemical Toxicology 1994, 17(1), 69-73,
Tetrahedron letters 1992, 33(21), 2961-4,
Fundamental and Applied Toxicology 1995, 26(2), 239-45,
Toxicology 1992, 76(1), 79-87,
J. Org. Chem. 1991, 56(26), 7328-7331,
Methods in Enzymology 1995, 251 (Biothiols, part A), 167-173,
U.S. Pat. No. 2,930,799,
J. Org. Chem. 1981, 46(10), 2072-9.

It is a further object of the present invention to provide a method for permanently reshaping the hair using a reducing composition comprising at least one compound of formula (I) and/or (II), and/or at least one organic or mineral salt of said compound.

The permanent hair reshaping method according to the present invention comprises:
An application step onto the hair of a reducing composition according to the present invention, to reduce the keratin disulfide bonds;
An oxidizing fixing step, to form said bonds again, by application onto the hair of an oxidizing composition or by contacting the hair with atmospheric oxygen.

The hair is set using mechanic means well known to those skilled in the art.

To make a permanent waving, curlers are used preferably, the reducing composition being applied before or after the hair set means and the oxidizing fixing composition being applied after the reducing composition, with or without an intermediate or a subsequent step to rinse out or to apply an intermediate composition.

The reducing composition according to the present invention is preferably applied to wet hair, which have been previously wrapped round curlers of 2 to 30 mm diameter. The composition may also be applied as hair rolling process progresses. The reducing composition is then left generally for a period of time comprised between 5 and 60 minutes, preferably between 15 and 45 minutes to act, then has to be rinsed out thoroughly. The oxidizing composition is then applied to wrapped hair, said oxidizing composition enabling to re-form the keratin disulfide bonds, for an application time from 2 to 10 minutes. After having removed the curlers, the hair is rinsed out thoroughly.

Once the reducing composition has been applied, the hair may also undergo a thermal treatment by heating at a temperature comprised between 30 and 60° C. for all or part of the application time. In practice, this step may be conducted using a helmet hair drier, a hair drier, an infrared radiation drier and other usual heating devices.

A heating iron may also be used, the temperature of which varies between 60 and 220° C., preferably between 120 and 220° C., both as heating means and as hair setting means, the heating iron being used between the application step of the reducing composition and the fixing step.

To carry out the uncurling or the straightening of the hair, a reducing composition according to the present invention has to be applied onto the hair, then the hair undergoes a mechanical reshaping, so as to get fixed in its new shape by a smoothing operation with wide toothed comb, with back of the comb or back of the hand. After generally 5 to 60 minutes application time, preferably 15 to 45 minutes, the hair has to be smoothed down once again, then carefully rinsed out, and the oxidizing composition is applied such as defined hereinbefore, which has to be left on for about 2 to 10 minutes before rinsing the hair thoroughly.

The oxidizing composition may be any oxidizing composition traditionally used to permanently reshape hair and which contains an oxidizing agent preferably selected from hydrogen peroxide, alkaline bromates, persalts, polythionates and a mixture made of alkaline bromate and persalt. Peroxide hydrogen concentration may range from 1 to 20 volumes and preferably from 1 to 10, alkaline bromate concentration may range from 2 to 12% and persalt concentration from 0.1 to 15% by weight, as compared to the total weight of the oxidizing composition.

The oxidizing composition pH is generally between 2 and 10.

As explained before, the oxidizing composition may be applied immediately or later.

According to a particular embodiment of the method according to the present invention, the smoothing of the hair may also be done, in all or part, with a heating iron at a temperature between 60 and 220° C., and preferably between 120 and 200° C.

It is another object of the present invention to provide a preparation kit, in particular to permanently reshape the hair, comprising, in a first compartment, as reducing composition, a reducing composition according to the present invention containing one or more compounds of formula (I) and/or (II), such as defined hereinbefore an/or one or more organic and mineral salts thereof, and in a second compartment, an oxidizing composition.

Generally, the oxidizing composition comprises at least one oxidizing agent selected from the group consisting of hydrogen peroxide, alkaline bromates, persalts, polythionates and a mixture of alkaline bromate and persalt.

It is another object of the present invention to provide as new compound a dicarboxydithiol having the general formula (III) or the general formula (IV) as follows: 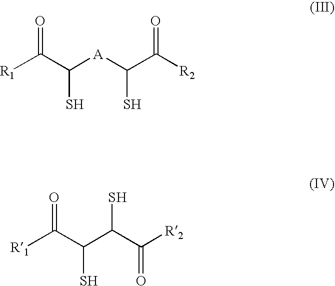
wherein:
A, R₁, R₂, R′₁, and R′₂ have the same meanings as above, except the compounds wherein:
   (i) R₁₁ represents OH and R′₂ represents OR₃, R₃ being a linear or branched C₁-C₅ alkyl radical, or R′₂ represents NR₄R₅, R₄ being a hydrogen atom and R₅ a branched C₃-C₅ alkyl radical;
   (ii) R′₁ and R′₂ represent an amino radical;
   (iii) R′₁ and R′₂ represent a OR₃ radical, where R₃ represents a branched or linear C₁-C₅ alkyl radical, as well as 2-methoxyethyl and 2-ethoxyethyl radicals;
   (iv) A represents CH₂ and R₁ and R₂ represent an amino radical;
   (v) A represents CH₂ and R₁ and R₂ represent a OR₃ radical, where R₃ represents a butyl radical;
   (vi) A represents (CH₂)₂, R₁ represents OH and R₂ represents NR₄R₅, R₄ being a hydrogen atom and R₅ being a hydrogen atom;
   (vii) A represents (CH₂)₂ and R₁ and R₂ represent a dimethylamino radical;
   (viii) A represents (CH₂)₂ and R₁ and R₂ represent a OR₃ radical, where R₃ represents a methyl radical;
or a mineral or organic salt of said compound of formula (III) or (IV).

Amongst the compounds of general formula (III) or (IV), the preferred compounds are as follows:
2,3-dimercapto-4-methylamino-4-oxo-butanoic acid,
2,3-dimercapto-4-ethylamino-4-oxo-butanoic acid,
2,3-dimercapto-4-propylamino-4-oxo-butanoic acid,
2,3-dimercapto-4-butylamino-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-N,N′-dimethyl-butanediamide,
2,3-dimercapto-N,N′-diethyl-butanediamide,
2,3-dimercapto-N,N′-bis(2-hydroxyethyl)-butanediamide,
2,3-dimercapto-N,N′-bis(2-hydroxypropyl)-butanediamide,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-butylamino-5-oxo-pentanoic acid,
2,4-dimercapto-5-[(1-methylethyl)amino]-5-oxo-pentanoic acid,
2,4-dimercapto-5-[(2-methylpropyl)amino]-5-oxo-pentanoic acid,
2,4-dimercapto-5-[(3-methylbutyl)amino]-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-pentanedioic acid, monomethylester,
2,4-dimercapto-pentanedioic acid, monoethylester,
2,4-dimercapto-pentanedioic acid, monopropylester,
2,4-dimercapto-pentanedioic acid, mono(1-methylethyl)ester,
2,4-dimercapto-N,N′-dimethyl-pentanediamide,
2,4-dimercapto-N,N′-diethyl-pentanediamide,
2,4-dimercapto-N,N′-bis(2-hydroxyethyl)-pentanediamide,
2,4-dimercapto-N,N′-bis(2-hydroxypropyl)-pentanediamide,
2,4-dimercapto-pentanedioic acid, dimethylester,
2,4-dimercapto-pentanedioic acid, diethylester,
2,4-dimercapto-pentanedioic acid, dipropylester,
2,5-dimercapto-6-methylamino-6-oxo-hexanoic acid,
2,5-dimercapto-6-ethylamino-6-oxo-hexanoic acid,
2,5-dimercapto-6-propylamino-6-oxo-hexanoic acid,
2,5-dimercapto-6-[(2-hydroxyethyl)amino]-6-oxo-hexanoic acid,
2,5-dimercapto-6-[(2-hydroxypropyl)amino]-6-oxo-hexanoic acid,
2,5-dimercapto-6-[(2-dimethylaminoethyl)amino]-6-oxo-hexanoic acid,
2,5-dimercapto-6-[(3-dimethylaminopropyl)amino]-6-oxo-hexanoic acid
2,5-dimercapto-hexanedioic acid, monomethylester,
2,5-dimercapto-hexanedioic acid, monoethylester,
2,5-dimercapto-hexanedioic acid, monopropylester,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
2,5-dimercapto-hexanedioic acid, diethylester,
2,5-dimercapto-hexanedioic acid, dipropylester,
2,6 dimercapto-7-methylamino-7- oxo-heptanedioic acid,
2,6- dimercapto-7-ethylamino-7- oxo-heptanedioic acid,
2,6- dimercapto-7-propylamino-7- oxo-heptanedioic acid,
2,6-dimercapto-7-[(2-hydroxyethyl)amino]-7- oxo-heptanedioic acid,
2,6-dimercapto-7-[(2-hydroxypropyl)amino]-7- oxo-heptanedioic acid,
2,6-dimercapto-7-[(2-dimethylaminoethyl)amino]-7- oxo-heptanedioic acid,
2,6-dimercapto-7-[(3-dimethylaminopropyl)amino]-7- oxo-heptanedioic acid,
2,6-dimercapto-heptanedioic acid, monomethylester,
2,6-dimercapto-heptanedioic acid, monoethylester,
2,6-dimercapto-heptanedioic acid, monopropylester,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
2,6-dimercapto-heptanedioic acid, dimethylester,
2,6-dimercapto-heptanedioic acid, diethylester,
2,6-dimercapto-heptanedioic acid, dipropylester,
2,7-dimercapto-8-methylamino-8- oxo-octanedioic acid,
2,7-dimercapto-8-ethylamino-8- oxo-octanedioic acid,
2,7-dimercapto-8-propylamino-8-oxo-octanedioic acid,
2,7-dimercapto-8-[(2-hydroxyethyl)amino]-8-oxo-octanedioic acid,
2,7-dimercapto-8-[(2-hydroxypropyl)amino]-8-oxo-octanedioic acid,
2,7-dimercapto-8-[(2-dimethylaminoethyl)amino]-8-oxo-octanedioic acid,
2,7-dimercapto-8-[(3-dimethylaminopropyl)amino]-8-oxo-octanedioic acid,
2,8-dimercapto-octanedioic acid, monomethylester,
2,8-dimercapto-octanedioic acid, monoethylester,
2,8-dimercapto-octanedioic acid, monopropylester,
2,8-dimercapto-heptanediamide,
2,8-dimercapto-N,N′-dimethyl-heptanediamide, 2,8-dimercapto-N,N′-bis(2-hydroxyethyl)-heptanediamide,
2,8-dimercapto-N,N′-bis(2-hydroxypropyl)-heptanediamide,
2,8-dimercapto-heptanedioic acid, dimethylester,
2,8-dimercapto-heptanedioic acid, diethylester,
2,8-dimercapto-heptanedioic acid, dipropylester,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-propylamino-5-oxo-pentanoic acid,
2,4-dimercapto-3-oxa-5-butylamino-5-oxo-pentanoic acid,
2,4-dimercapto-3-oxa-5-[(1-methylethyl)amino]-5-oxo-pentanoic acid,
2,4-dimercapto-3-oxa-5-[(2-methylpropyl)amino]-5-oxo-pentanoic acid,
2,4-dimercapto-3-oxa-5-[(3-methylbutyl)amino]-5-oxo-pentanoic acid,
2,4-dimercapto-3-oxa-5-[(2-hydroxyethyl)amino]-5-oxo-pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanedioic acid, monomethylester,
2,4-dimercapto-3-oxa-pentanedioic acid, monoethylester,
2,4-dimercapto-3-oxa-pentanedioic acid, monopropylester,
2,4-dimercapto-3-oxa-pentanedioic acid, mono(1-methylethyl)ester,
2,4-dimercapto-3-oxa-pentanediamide,
2,4-dimercapto-3-oxa-N,N′-dimethyl-pentanediamide,
2,4-dimercapto-3-oxa-N,N′-diethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,4-dimercapto-3-oxa-pentanedioic acid, dimethylester,
2,4-dimercapto-3-oxa-pentanedioic acid, diethylester,
2,4-dimercapto-3-oxa-pentanedioic acid, dipropylester,
2,4-dimercapto-3-oxa-pentanedioic acid, dibutylester,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-propylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-7-[(2-dimethylaminoethyl)amino]-7-oxo-heptanedioic acid,
2,6-dimercapto-4-thia-7-[(3-dimethylaminopropyl)amino]-7-oxo-heptanedioic acid,
2,6-dimercapto-4-thia-heptanedioic acid, monomethylester,
2,6-dimercapto-4-thia-heptanedioic acid, monoethylester,
2,6-dimercapto-4-thia-heptanedioic acid, monopropylester,
2,6-dimercapto-4-thia-heptanediamide,
2,6-dimercapto 4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-thia-N,N′-bis(2-hydroxypropyl)-heptanediamide,
2,6-dimercapto-4-thia-heptanedioic acid, dimethylester,
2,6-dimercapto-4-thia-heptanedioic acid, diethylester,
2,6-dimercapto-4-thia-heptanedioic acid, dipropylester,
N-(carboxy-mercapto-methyl)-2-mercapto-malonamic acid,
[(carboxy-mercapto-methyl)-methyl-amino]-mercapto-acetic acid.

The following compounds are especially preferred:
2,3-dimercapto-4-methylamino-4- oxo-butanoic acid,
2,3-dimercapto-4-ethylamino-4- oxo-butanoic acid,
2,3-dimercapto-4-propylamino-4- oxo-butanoic acid,
2,3-dimercapto-4-butylamino-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-N,N′-dimethyl-butanediamide,
2,3-dimercapto-N,N′-bis(2-hydroxyethyl)-butanediamide,
2,3-dimercapto-N,N′-bis(2-hydroxypropyl)-butanediamide,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-N,N′-dimethyl-pentanediamide,
2,4-dimercapto-N,N′-bis(2-hydroxyethyl)-pentanediamide,
2,4-dimercapto-N,N′-bis(2-hydroxypropyl)-pentanediamide,
2,5-dimercapto-6-methylamino-6- oxo-hexanoic acid,
2,5-dimercapto-6-ethylamino-6- oxo-hexanoic acid,
2,5-dimercapto-6-[(2-hydroxyethyl)amino]-6- oxo-hexanoic acid,
2,5-dimercapto-6-[(2-hydroxypropyl)amino]-6- oxo-hexanoic acid,
2,5-dimercapto-6-[(2-dimethylaminoethyl)amino]-6- oxo-hexanoic acid,
2,5-dimercapto-6-[(3-dimethylaminopropyl)amino]-6- oxo-hexanoic acid,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
2,6-dimercapto-7-methylamino-7-oxo-heptanedioic acid,
2,6-dimercapto-7-ethylamino-7-oxo-heptanedioic acid,
2,6-dimercapto-7-[(2-hydroxyethyl)amino]-7-oxo-heptanedioic acid,
2,6-dimercapto-7-[(2-hydroxypropyl)amino]-7-oxo-heptanedioic acid,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
2,7-dimercapto-8-methylamino-8-oxo-octanedioic acid,
2,7-dimercapto-8-ethylamino-8-oxo-octanedioic acid,
2,7-dimercapto-8-[(2-hydroxyethyl)amino]-8-oxo-octanedioic acid,
2,7-dimercapto-8-[(2-hydroxypropyl)amino]-8-oxo-octanedioic acid,
2,8-dimercapto-heptanediamide,
2,8-dimercapto-N,N′-dimethyl-heptanediamide,
2,8-dimercapto-N,N′-bis(2-hydroxyethyl)-heptanediamide,
2,8-dimercapto-N,N′-bis(2-hydroxypropyl)-heptanediamide,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-[(2-hydroxyethyl)amino]-5-oxo-pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanediamide,
2,4-dimercapto-3-oxa-N,N′-dimethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-heptanediamide,
2,6-dimercapto-4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-4-thia-N,N′-bis(2-hydroxypropyl)-heptanediamide.

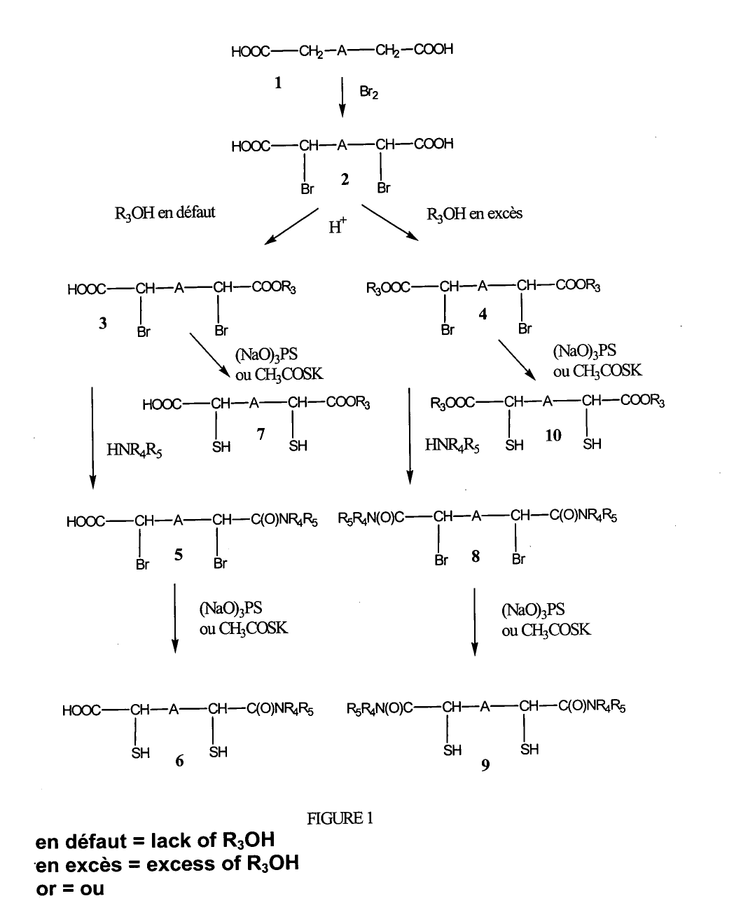

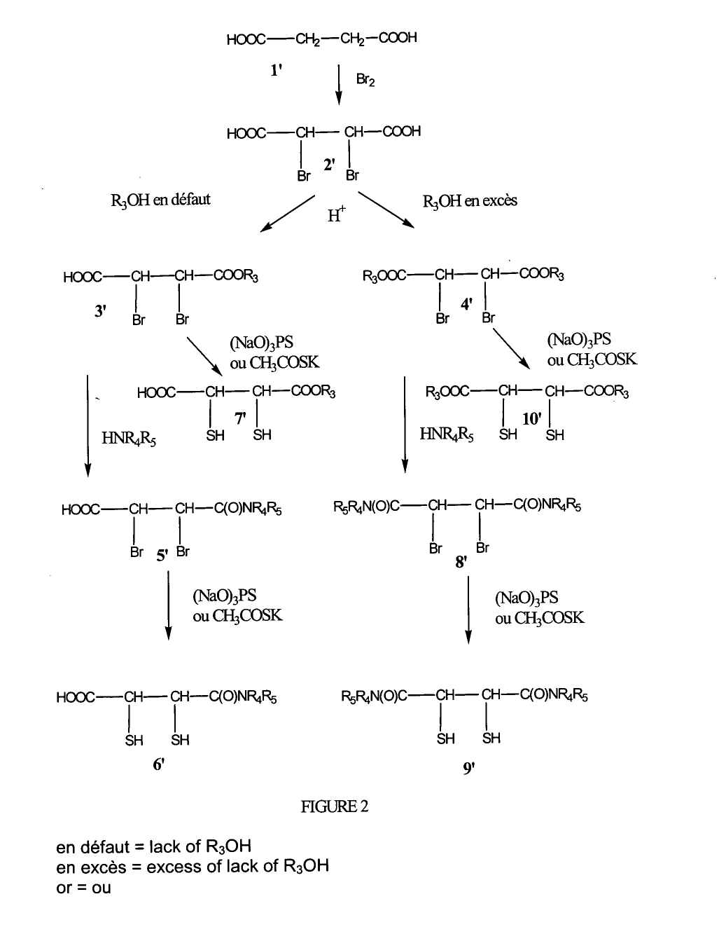

The new compounds of general formula (III) and (IV) may be prepared in the same way as the procedures described in the above references. They also may be prepared according to the synthesis scheme given hereinafter, by reference to FIGS. 1 and 2.

FIG. 1 represents the synthesis scheme for the new compounds of general formula (III).

FIG. 2 represents the synthesis scheme for the new compounds of general formula (IV).

Carboxylic diacid of formula (1) (respectively of formula (1′)) is brominated under the usual conditions, for example with phosphorus pentachloride or thionyl chloride in catalytic amounts to give dibrominated acid of formula (2) (respectively of formula (2′)).

This is classically esterified with a primary alcohol of formula R₃—OH, for example with a strong acid, such as sulfuric acid, in catalytic amounts, to give the compounds of formula (3) and (4) (respectively of formula (3′) and (4′)).

A thiophosphate is reacted, preferably sodium thiophosphate, with these compounds of formula (3) and (4) (respectively of formula (3′) and (4′)) in an aprotic dipolar solvent such as tetrahydrofurane or dimethylformamide, or in a protic solvent such as methanol, at a temperature between ambient temperature and reflux temperature of the selected solvent, then hydrolysis is carried out with a diluted solution of a strong acid such as hydrochloric acid or sulfuric acid. These compounds of formula (3) and (4) (respectively of formula (3′) and (4′)) may also be reacted with thioacetic acid, preferably its potassium salt, then hydrolysis is carried out preferably in a basic medium. The compounds according to the present invention of formula (7) and (10) (respectively of formula (7′) and (10′)) are thus respectively produced.

The corresponding amides of formula (5) and (8) (respectively of formula (5′) and (8′)) may also be prepared from the compounds of formula (3) and (4) (respectively of formula (3′) and (4′)), by reaction with an amine of formula HNR₄R₅ in the conditions normally employed to convert esters into amides.

A thiophosphate, preferably sodium thiophosphate, is then reacted with these compounds of formula (5) and (8) in a dipolar aprotic solvent, such as tetrahydrofurane or dimethylformamide, or in a protic solvent such as methanol at a temperature between ambient temperature and reflux temperature of the selected solvent, then hydrolysis is conducted with a diluted solution of a strong acid such as hydrochloric acid or sulfuric acid. These compounds of formula (5) and (8) (respectively of formula (5′) and (8′)) may also be reacted with thioacetic acid, preferably its potassium salt, then hydrolysis is carried out preferably in a basic medium. The compounds according to the present invention of formula (6) and (9) (respectively of formula (6′) and (9′)) are thus respectively produced.

The present invention will be better understood by reference to the following non limitative examples which represent preferred embodiments for the compositions according to the present invention.

### COMPOSITION EXAMPLES

### Example 1

A reducing composition to permanently reshape hair according to the present invention is prepared par mixing the following compounds together:

| | |
|---|---|
| 2, 3-dimercapto-4-ethylamino-4-oxo-butanoic acid: | 12.5 g |
| 20% ammonia: | qs pH 8.5 |
| demineralized water: | qs 100 g |

This composition is applied onto wet hair, previously rolled up on set curlers. The composition has to be left on for about 15 minutes, then the hair is blow dried with a hair dryer for 5 minutes and thoroughly rinsed out with water.

The following oxidizing composition is then applied:

| | |
|---|---|
| hydrogen peroxide (200 vol.): | 4.8 g |
| citric acid: | qs pH 3 |
| demineralized water: | qs 100 g |

The oxidizing composition leaves on for 5 minutes, then has to be rinsed out thoroughly with water. Curlers are then removed, and hair is dried under a hood. As a result, the hair curling is bouncy and dynamic.

### Example 2

A reducing composition to permanently reshape hair according to the present invention is prepared par mixing the following ingredients together:

| | |
|---|---|
| 2, 3-dimercapto-4-[(2-hydroxypropyl)amino]-4-oxo-butanoic | 16.7 g |
| acid | |
| 20% ammonia: | qs pH 8.5 |
| demineralized water: | qs 100 g |

This composition is applied onto wet hair, previously rolled up on set curlers. The composition has to be left on for about 15 minutes, then the hair is blow dried with a hair dryer for 5 minutes and thoroughly rinsed out with water.

The following oxidizing composition is then applied:

| | |
|---|---|
| hydrogen peroxide (200 vol.): | 4.8 g |
| citric acid: | qs pH 3 |
| demineralized water: | qs 100 g |

The oxidizing composition leaves on for 5 minutes, then has to be rinsed out thoroughly with water. Curlers are removed and hair is dried under a hood. As a result, the hair curling is bouncy and dynamic.

1. An aqueous reducing composition, for the first step of a process to permanently reshape the hair, comprising in a cosmetically acceptable medium, as reducing agent, one or more dicarboxydithiols of general formula (I) and/or of general formula (II): 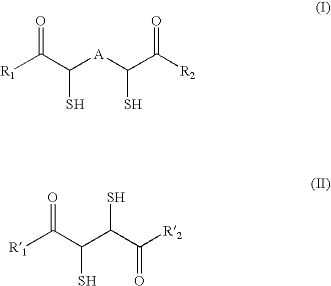
wherein:
R₁, R₂, R₁₁, R₁₂ independently represent OR₃ or NR₄R₅, where R₃ represents H or a linear or branched C₁-C₅ alkyl group optionally substituted by one or two OR₇ radicals, where R₇ represents H or a C₁-C₃ alkoxy radical,
R₄, R₅ are independently H or a linear or branched C₁-C₅ alkyl group optionally substituted by one or two radicals selected from hydroxy, methoxy, ethoxy, methylamino, ethylamino and dimethylamino radicals,
R₁ and R₂ not being simultaneously a hydroxyl radical and R₁₁ and R₁₂ not being simultaneously a hydroxyl radical;
A either represents (CH₂)ₙ, where n is an integer ranging from 1 to 4, or a —(CH₂)ₘ—X—(CH₂)ₚ— radical, where m is 1 or 2, p is 1 or 2 and X represents O, S, NHCO or NR₆, where R₆ represents H or CH₃,
and/or one or more organic and mineral salts thereof.

2. A composition according to claim 1, characterized in that dicarboxydithiol(s) of general formula (I) and/or of general formula (II) are selected from the group consisting of following compounds:
2,3-dimercapto-4-methylamino-4-oxo-butanoic acid,
2,3-dimercapto-4-ethylamino-4-oxo-butanoic acid,
2,3-dimercapto-4-propylamino-4-oxo-butanoic acid,
2,3-dimercapto-4-butylamino-4-oxo-butanoic acid,
2,3-dimercapto-4-[(1-methylethyl)amino]-4-oxo-butanoic acid,
2,3-dimercapto-4-[(2-methylpropyl)amino]-4-oxo-butanoic acid,
2,3-dimercapto-4-[(3-methylbutyl)amino]-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-butanedioic acid, monomethylester,
2,3-dimercapto-butanedioic acid, monoethylester,
2,3-dimercapto-butanedioic acid, monopropylester,
2,3-dimercapto-butanedioic acid, mono(1-methylethyl)ester,
2,3-dimercapto-butanedioic acid, monobutylester,
2,3-dimercapto-butanedioic acid, mono(2-methylpropyl)ester,
2,3-dimercapto-butanedioic acid, monopentylester,
2,3-dimercapto-butanedioic acid, mono(3-methylbutyl)ester,
2,3-dimercapto-butanediamide,
2,3-dimercapto-N,N′-dimethyl-butanediamide,
2,3-dimercapto-N,N′-diethyl-butanediamide,
2,3-dimercapto-N,N′-bis(2-hydroxyethyl)-butanediamide,
2,3-dimercapto-N,N′-bis(2-hydroxypropyl)-butanediamide,
2,3-dimercapto-butanedioic acid, dimethylester,
2,3-dimercapto-butanedioic acid, diethylester,
2,3-dimercapto-butanedioic acid, dipropylester,
2,3-dimercapto-butanedioic acid, bis(1-methylethyl)ester,
2,3-dimercapto-butanedioic acid, dibutylester,
2,3-dimercapto-butanedioic acid, bis(2-methylpropyl)ester,
2,3-dimercapto-butanedioic acid, bis(1,1-dimethylethyl)ester,
2,3-dimercapto-butanedioic acid, bis(1-methylpropyl)ester,
2,3-dimercapto-butanedioic acid, bis(3-methylbutyl)ester,
2,3-dimercapto-butanedioic acid, bis(1-ethylpropyl)ester,
2,3-dimercapto-butanedioic acid, bis(2-methoxyethyl)ester,
2,3-dimercapto-butanedioic acid, bis(2-ethoxyethyl)ester,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-butylamino-5-oxo-pentanoic acid,
2,4-dimercapto-5-[(1-methylethyl)amino]-5-oxo-pentanoic acid,
2,4-dimercapto-5-[(2-methylpropyl)amino]-5-oxo-pentanoic acid,
2,4-dimercapto-5-[(3-methylbutyl)amino]-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-pentanedioic acid, monomethylester,
2,4-dimercapto-pentanedioic acid, monoethylester,
2,4-dimercapto-pentanedioic acid, monopropylester,
2,4-dimercapto-pentanedioic acid, mono(1-methylethyl)ester,
2,4-dimercapto-pentanediamide,
2,4-dimercapto-N,N′-dimethyl-pentanediamide,
2,4-dimercapto-N,N′-diethyl-pentanediamide,
2,4-dimercapto-N,N′-bis(2-hydroxyethyl)-pentanediamide,
2,4-dimercapto-N,N′-bis(2-hydroxypropyl)-pentanediamide,
2,4-dimercapto-pentanedioic acid, dimethylester,
2,4-dimercapto-pentanedioic acid, diethylester,
2,4-dimercapto-pentanedioic acid, dipropylester,
2,4-dimercapto-pentanedioic acid, dibutylester,
5- carbamoyl-2,5-dimercapto-valeric acid,
2,5-dimercapto-6-methylamino-6- oxo-hexanoic acid,
2,5-dimercapto-6-ethylamino-6- oxo-hexanoic acid,
2,5-dimercapto-6-propylamino-6- oxo-hexanoic acid,
2,5-dimercapto-6-[(2-hydroxyethyl)amino]-6- oxo-hexanoic acid,
2,5-dimercapto-6-[(2-hydroxypropyl)amino]-6- oxo-hexanoic acid,
2,5-dimercapto-6-[(2-dimethylaminoethyl)amino]-6- oxo-hexanoic acid,
2,5-dimercapto-6-[(3-dimethylaminopropyl)amino]-6- oxo-hexanoic acid,
2,5-dimercapto-hexanedioic acid, monomethylester,
2,5-dimercapto-hexanedioic acid, monoethylester,
2,5-dimercapto-hexanedioic acid, monopropylester,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
2,5-dimercapto-N,N, N′,N′-tetramethyl-hexanediamide,
l'acide 2,5-dimercapto-hexanedioic acid, dimethylester,
l'acide 2,5-dimercapto-hexanedioic acid, diethylester,
l'acide 2,5-dimercapto-hexanedioic acid, dipropylester,
2,6-dimercapto-7-methylamino-7-oxo-heptanedioic acid,
2,6-dimercapto-7-ethylamino-7-oxo-heptanedioic acid,
2,6-dimercapto-7-propylamino-7-oxo-heptanedioic acid,
2,6-dimercapto-7-[(2-hydroxyethyl)amino]-7-oxo-heptanedioic acid,
2,6-dimercapto-7-[(2-hydroxypropyl)amino]-7-oxo-heptanedioic acid,
2,6-dimercapto-7-[(2-dimethylaminoethyl)amino]-7-oxo-heptanedioic acid,
2,6-dimercapto-7-[(3-dimethylaminopropyl)amino]-7-oxo-heptanedioic acid,
2,6-dimercapto-heptanedioic acid, monomethylester,
2,6-dimercapto-heptanedioic acid, monoethylester,
2,6-dimercapto-heptanedioic acid, monopropylester,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
2,6-dimercapto-heptanedioic acid, dimethylester,
2,6-dimercapto-heptanedioic acid, diethylester,
2,6-dimercapto-heptanedioic acid, dipropylester,
2,7-dimercapto-8-methylamino-8-oxo-octanedioic acid,
2,7-dimercapto-8-ethylamino-8-oxo-octanedioic acid,
2,7-dimercapto-8-propylamino-8-oxo-octanedioic acid,
2,7-dimercapto-8-[(2-hydroxyethyl)amino]-8-oxo-octanedioic acid,
2,7-dimercapto-8-[(2-hydroxypropyl)amino]-8-oxo-octanedioic acid,
l'acide 2,7-dimercapto-8-[(2-dimethylaminoethyl)amino]-8-oxo-octanedioic acid,
2,7-dimercapto-8-[(3-dimethylaminopropyl)amino]-8-oxo-octanedioic acid,
2,8-dimercapto-octanedioic acid, monomethylester,
2,8-dimercapto-octanedioic acid, monoethylester,
2,8-dimercapto-octanedioic acid, monopropylester,
2,8-dimercapto-heptanediamide,
2,8-dimercapto-N,N′-dimethyl-heptanediamide,
2,8-dimercapto-N,N′-bis(2-hydroxyethyl)-heptanediamide,
2,8-dimercapto-N,N′-bis(2-hydroxypropyl)-heptanediamide,
2,8-dimercapto-heptanedioic acid, dimethylester,
2,8-dimercapto-heptanedioic acid, diethylester,
2,8-dimercapto-heptanedioic acid, dipropylester,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-propylamino-5-oxo-pentanoic acid,
2,4-dimercapto-3-oxa-5-butylamino-5-oxo-pentanoic acid,
2,4-dimercapto-3-oxa-5-[(1-methylethyl)amino]-5-oxo-pentanoic acid,
2,4-dimercapto-3-oxa-5-[(2-methylpropyl)amino]-5-oxo-pentanoic acid,
2,4-dimercapto-3-oxa-5-[(3-methylbutyl)amino]-5-oxo-pentanoic acid,
2,4-dimercapto-3-oxa-5-[(2-hydroxyethyl)amino]-5-oxo-pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanedioic acid, monomethylester,
2,4-dimercapto-3-oxa-pentanedioic acid, monoethylester,
2,4-dimercapto-3-oxa-pentanedioic acid, monopropylester,
2,4-dimercapto-3-oxa-pentanedioic acid, mono(1-methylethyl)ester,
2,4-dimercapto-3-oxa-pentanediamide,
2,4-dimercapto-3-oxa-N,N′-dimethyl-pentanediamide,
2,4-dimercapto-3-oxa-N,N′-diethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,4-dimercapto-3-oxa-pentanedioic acid, dimethylester,
2,4-dimercapto-3-oxa-pentanedioic acid, diethylester,
2,4-dimercapto-3-oxa-pentanedioic acid, dipropylester,
2,4-dimercapto-3-oxa-pentanedioic acid, dibutylester,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-propylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-7-[(2-dimethylaminoethyl)amino]-7-oxo-heptanedioic acid,
2,6-dimercapto-4-thia-7-[(3-dimethylaminopropyl)amino]-7-oxo-heptanedioic acid,
2,6-dimercapto-4-thia-heptanedioic acid, monomethylester,
2,6-dimercapto-4-thia-heptanedioic acid, monoethylester,
2,6-dimercapto-4-thia-heptanedioic acid, monopropylester,
2,6-dimercapto-4-thia-heptanediamide,
2,6-dimercapto-4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-4-thia-N,N′-bis(2-hydroxypropyl)-heptanediamide,
2,6-dimercapto-4-thia-heptanedioic acid, dimethylester,
2,6-dimercapto-4-thia-heptanedioic acid, diethylester,
2,6-dimercapto-4-thia-heptanedioic acid, dipropylester,
N-(carboxy-mercapto-methyl)-2-mercapto-malonamic acid,
[(carboxy-mercapto-methyl)-methyl-amino]-mercapto-acetic acid.

3. A composition according to claim 2, characterized in that the dicarboxythiol(s) of general formula (I) and/or of general formula (II) are selected from the group consisting of following compounds:
2,3-dimercapto-4-methylamino-4-oxo-butanoic acid,
2,3-dimercapto-4-ethylamino-4-oxo-butanoic acid,
2,3-dimercapto-4-propylamino-4-oxo-butanoic acid,
2,3-dimercapto-4-butylamino-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-butanediamide,
2,3-dimercapto-N,N′-dimethyl-butanediamide,
2,3-dimercapto-N,N′-bis(2-hydroxyethyl)-butanediamide,
2,3-dimercapto-N,N′-bis(2-hydroxypropyl)-butanediamide,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-pentanediamide,
2,4-dimercapto-N,N′-dimethyl-pentanediamide,
2,4-dimercapto-N,N′-bis(2-hydroxyethyl)-pentanediamide,
2,4-dimercapto-N,N′-bis(2-hydroxypropyl)-pentanediamide,
5- carbamoyl-2,5-dimercapto-valeric acid,
2,5-dimercapto-6-methylamino-6- oxo-hexanoic acid,
2,5-dimercapto-6-ethylamino-6- oxo-hexanoic acid,
2,5-dimercapto-6-[(2-hydroxyethyl)amino]-6- oxo-hexanoic acid,
2,5-dimercapto-6-[(2-hydroxypropyl)amino]-6- oxo-hexanoic acid,
2,5-dimercapto-6-[(2-dimethylaminoethyl)amino]-6- oxo-hexanoic acid,
2,5-dimercapto-6-[(3-dimethylaminopropyl)amino]-6- oxo-hexanoic acid,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
2,6-dimercapto-7-methylamino-7-oxo-heptanedioic acid,
2,6-dimercapto-7-ethylamino-7-oxo-heptanedioic acid,
2,6-dimercapto-7-[(2-hydroxyethyl)amino]-7-oxo-heptanedioic acid,
2,6-dimercapto-7-[(2-hydroxypropyl)amino]-7-oxo-heptanedioic acid,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
2,7-dimercapto-8-methylamino-8-oxo-octanedioic acid,
2,7-dimercapto-8-ethylamino-8-oxo-octanedioic acid,
2,7-dimercapto-8-[(2-hydroxyethyl)amino]-8-oxo-octanedioic acid,
2,7-dimercapto-8-[(2-hydroxypropyl)amino]-8-oxo-octanedioic acid,
2,8-dimercapto-heptanediamide,
2,8-dimercapto-N,N′-dimethyl-heptanediamide,
2,8-dimercapto-N,N′-bis(2-hydroxyethyl)-heptanediamide,
2,8-dimercapto-N,N′-bis(2-hydroxypropyl)-heptanediamide,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-[(2-hydroxyethyl)amino]-5-oxo- pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanediamide,
2,4-dimercapto-3-oxa-N,N′-dimethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-heptanediamide,
2,6-dimercapto 4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-4-thia-N,N′-bis(2-hydroxypropyl)-heptanediamide.

4. A composition according to any of the preceding claims, characterized in that the dicarboxydithiol(s) of general formula (I) and/or (II), and/or their organic and mineral salts, are present in a quantity in the range between 0.05 and 30%, preferably between 1 and 20% by weight, as compared to the total weight of the composition.

5. A composition according to any of the preceding claims, characterized in that pH is between 4 and 11, preferably between 6 and 10.

6. A composition according to claim 5, characterized in that pH is balanced with an alkaline agent selected from the group consisting of ammonia, monoethanolamine, diethanolamine, 1,3-propanediamine, alkaline or ammonium carbonate or bicarbonate, organic carbonate, preferably guanidine carbonate and alkaline hydroxide, or with an acidifying agent selected from the group consisting of hydrochloric acid, acetic acid, lactic acid, oxalic acid or boric acid, or with a borate, phosphate or TRIS-buffer.

7. A composition according to any of the preceding claims, characterized in that it also comprises one or more other reducing agents selected from thioglycolic acid, thiolactic acid, glycerol monothioglycolate, cysteamine, N-acetyl-cysteamine, N-propionyl-cysteamine, cysteine, N-acetyl-cysteine, thiomalic acid, pantheteine, 2,3-dimercaptosuccinic acid, alkali metal or alkaline-earth metal sulfites or bisulfites, N-(mercaptoalkyl)-ω-hydroxyalkylamides, N-mono or N,N-dialkylmercapto-4-butyramides, aminomercapto-alkylamides, N-(mercaptoalkyl)succinamic acid derivatives and N-(mercaptoalkyl)succinimides derivatives, alkylamino mercaptoalkylamides, an azeotropic mixture made of 2-hydroxypropyl thioglyconate and (2-hydroxy-1-methyl)ethyl thioglycolate, mercaptoalkylaminoamides, N-mercapto-alkylalkanediamides and derivatives of formamidine sulfinic acid.

8. A composition according to any of the preceding claims, characterized in that it comprises at least one surfactant selected from the group consisting of alkyl sulfates, alkyl benzene sulfates, alkyl ether sulfates, alkyl sulfonates, quaternary ammonium salts, alkyl betaines, oxyethylene alkyl phenols, fatty acid alkanolamides, oxyethylene fatty acid esters, hydroxypropylethers.

9. A composition according to claim 8, characterized in that the surfactant(s) represent at most 30% by weight, preferably between 0.5 and 10% by weight, as compared to the total weight of the composition.

10. A composition according to any of the preceding claims, characterized in that it comprises a vehicle selected from the group consisting of water and a hydroalcoholic solution of a lower C₁-C₆ alcohol, preferably ethanol, isopropanol or butanol.

11. A composition according to any of the preceding claims, characterized in that it is in aqueous form, preferably as a lotion thickened or not, as a cream thickened or not, or as a gel.

12. A method for permanently reshaping the hair, which comprises:
an application step onto the hair of the reducing composition according to any one of claims 1 to 11, to reduce keratin disulfide bonds,
an oxidizing-fixing step, to re-form said bonds, by application onto the hair of an oxidizing composition or by contacting the hair with atmospheric oxygen.

13. A method according to claim 12, characterized in that the reducing composition is left on for 5 to 60 minutes, preferably for 15 to 45 minutes.

14. A method according to claim 13, characterized in that the oxidizing composition comprises at least one oxidizing agent selected from the group consisting of hydrogen peroxide, alkaline bromates, persalts, les polythionates and a mixture of alkaline bromate and persalt.

15. A method according to any one of claims 12 to 14, characterized in that it comprises, between the application step of the reducing composition onto the hair and the fixing step, a hair heating step with a heating iron at a temperature between 60 and 220° C. preferably between 120 and 200° C.

16. A kit to permanently reshape the hair, comprising in a first compartment a reducing composition such as defined in any one of claims 1 to 11, and in a second compartment an oxidizing composition.

17. A dicarboxydithiol, characterized in that it has the following general formula (III) or the following general formula (IV): 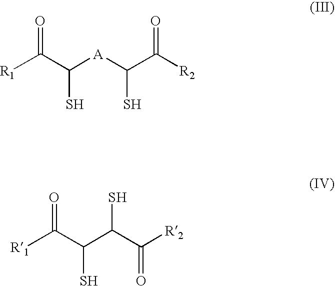
wherein:
A, R₁, R₂, R′₁, and R′₂ are such as defined in claim 1, except for the compounds wherein:
(i) R′ᵢ represents OH and R′₂ represents OR₃, R₃ being a linear or branched C₁-C₅-alkyl radical, or R′₂ represents NR₄R₅, R₄ being a hydrogen atom and R₅ a branched C₃-C₅ alkyl radical;
(ii) R′₁ and R′₂ represent an amino radical;
(iii) R′₁ and R′₂ represent a OR₃ radical, where R₃ represents a branched or linear C₁-C₅ alkyl radical as well as 2-methoxyethyl and 2-ethoxyethyl radicals;
(iv) A represents CH₂ and R₁ and R₂ represent an amino radical;
(v) A represents CH₂ and R₁ and R₂ represent a OR₃ radical, where R₃ represents a butyl radical;
(vi) A represents (CH₂)₂, R₁ represents OH and R₂ represents NR₄R₅, R₄ being a hydrogen atom and R₅ being a hydrogen atom;
(vii) A represents (CH₂)₂ and R₁ and R₂ represent a dimethylamino radical;
(viii) A represents (CH₂)₂ and R₁ and R₂ represent a OR₃ radical, where R₃ represents a methyl radical;
or a mineral or organic salt of said compound of formula (III) or (IV).

18. A dicarboxydithiol according to claim 17, characterized in that it is selected from the group consisting of the following compounds:
2,3-dimercapto-4-methylamino-4-oxo-butanoic acid,
2,3-dimercapto-4-ethylamino-4-oxo-butanoic acid,
2,3-dimercapto-4-propylamino-4-oxo-butanoic acid,
2,3-dimercapto-4-butylamino-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-N,N′-dimethyl-butanediamide,
2,3-dimercapto-N,N′-diethyl-butanediamide,
2,3-dimercapto-N,N′-bis(2-hydroxyethyl)-butanediamide,
2,3-dimercapto-N,N′-bis(2-hydroxypropyl)-butanediamide,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-butylamino-5-oxo-pentanoic acid,
2,4-dimercapto-5-[(1-methylethyl)amino]-5-oxo-pentanoic acid,
2,4-dimercapto-5-[(2-methylpropyl)amino]-5-oxo-pentanoic acid,
2,4-dimercapto-5-[(3-methylbutyl)amino]-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-pentanedioic acid, monomethylester,
2,4-dimercapto-pentanedioic acid, monoethylester,
2,4-dimercapto-pentanedioic acid, monopropylester,
2,4-dimercapto-pentanedioic acid, mono(1-methylethyl)ester,
2,4-dimercapto-N,N′-dimethyl-pentanediamide,
2,4-dimercapto-N,N′-diethyl-pentanediamide,
2,4-dimercapto-N,N′-bis(2-hydroxyethyl)-pentanediamide,
2,4-dimercapto-N,N′-bis(2-hydroxypropyl)-pentanediamide,
2,4-dimercapto-pentanedioic acid, dimethylester,
2,4-dimercapto-pentanedioic acid, diethylester,
2,4-dimercapto-pentanedioic acid, dipropylester,
2,5-dimercapto-6-methylamino-6- oxo-hexanoic acid,
2,5-dimercapto-6-ethylamino-6- oxo-hexanoic acid,
2,5-dimercapto-6-propylamino-6- oxo-hexanoic acid,
2,5-dimercapto-6-[(2-hydroxyethyl)amino]-6- oxo-hexanoic acid,
2,5-dimercapto-6-[(2-hydroxypropyl)amino]-6- oxo-hexanoic acid,
2,5-dimercapto-6-[(2-dimethylaminoethyl)amino]-6- oxo-hexanoic acid,
2,5-dimercapto-6-[(3-dimethylaminopropyl)amino]-6- oxo-hexanoic acid
2,5-dimercapto-hexanedioic acid, monomethylester,
2,5-dimercapto-hexanedioic acid, monoethylester,
2,5-dimercapto-hexanedioic acid, monopropylester,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
l'acide 2,5-dimercapto-hexanedioic acid, diethylester,
l'acide 2,5-dimercapto-hexanedioic acid, dipropylester,
2,6-dimercapto-7-methylamino-7-oxo-heptanedioic acid,
2,6-dimercapto-7-ethylamino-7-oxo-heptanedioic acid,
2,6-dimercapto-7-propylamino-7-oxo-heptanedioic acid,
2,6-dimercapto-7-[(2-hydroxyethyl)amino]-7-oxo-heptanedioic acid,
2,6-dimercapto-7-[(2-hydroxypropyl)amino]-7-oxo-heptanedioic acid,
2,6-dimercapto-7-[(2-dimethylaminoethyl)amino]-7-oxo-heptanedioic acid,
2,6-dimercapto-7-[(3-dimethylaminopropyl)amino]-7-oxo-heptanedioic acid,
2,6-dimercapto-heptanedioic acid, monomethylester,
2,6-dimercapto-heptanedioic acid, monoethylester,
2,6-dimercapto-heptanedioic acid, monopropylester,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
2,6-dimercapto-heptanedioic acid, dimethylester,
2,6-dimercapto-heptanedioic acid, diethylester,
2,6-dimercapto-heptanedioic acid, dipropylester,
2,7-dimercapto-8-methylamino-8-oxo-octanedioic acid,
2,7-dimercapto-8-ethylamino 8-oxo-octanedioic acid,
2,7-dimercapto-8-propylamino-8-oxo-octanedioic acid,
2,7-dimercapto-8-[(2-hydroxyethyl)amino]-8-oxo-octanedioic acid,
2,7-dimercapto-8-[(2-hydroxypropyl)amino]-8-oxo-octanedioic acid,
2,7-dimercapto-8-[(2-dimethylaminoethyl)amino]-8-oxo-octanedioic acid,
2,7-dimercapto-8-[(3-dimethylaminopropyl)amino]-8-oxo-octanedioic acid,
2,8-dimercapto-octanedioic acid, monomethylester,
2,8-dimercapto-octanedioic acid, monoethylester,
2,8-dimercapto-octanedioic acid, monopropylester,
2,8-dimercapto-heptanediamide,
2,8-dimercapto-N,N′-dimethyl-heptanediamide,
2,8-dimercapto-N,N′-bis(2-hydroxyethyl)-heptanediamide,
2,8-dimercapto-N,N′-bis(2-hydroxypropyl)-heptanediamide,
2,8-dimercapto-heptanedioic acid, dimethylester,
2,8-dimercapto-heptanedioic acid, diethylester,
2,8-dimercapto-heptanedioic acid, dipropylester,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-propylamino-5-oxo-pentanoic acid,
2,4-dimercapto-3-oxa-5-butylamino-5-oxo-pentanoic acid,
2,4-dimercapto-3-oxa-5-[(1-methylethyl)amino]-5-oxo-pentanoic acid,
2,4-dimercapto-3-oxa-5-[(2-methylpropyl)amino]-5-oxo-pentanoic acid,
2,4-dimercapto-3-oxa-5-[(3-methylbutyl)amino]-5-oxo-pentanoic acid,
2,4-dimercapto-3-oxa-5-[(2-hydroxyethyl)amino]-5-oxo-pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanedioic acid, monomethylester,
2,4-dimercapto-3-oxa-pentanedioic acid, monoethylester,
2,4-dimercapto-3-oxa-pentanedioic acid, monopropylester,
2,4-dimercapto-3-oxa-pentanedioic acid, mono(1-methylethyl)ester,
2,4-dimercapto-3-oxa-pentanediamide,
2,4-dimercapto-3-oxa-N,N′-dimethyl-pentanediamide,
2,4-dimercapto-3-oxa-N,N′-diethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,4-dimercapto-3-oxa-pentanedioic acid, dimethylester,
2,4-dimercapto-3-oxa-pentanedioic acid, diethylester,
2,4-dimercapto-3-oxa-pentanedioic acid, dipropylester,
2,4-dimercapto-3-oxa-pentanedioic acid, dibutylester,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-propylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-7-[(2-dimethylaminoethyl)amino]-7-oxo-heptanedioic acid,
2,6-dimercapto-4-thia-7-[(3-dimethylaminopropyl)amino]-7-oxo-heptanedioic acid,
2,6-dimercapto-4-thia-heptanedioic acid, monomethylester,
2,6-dimercapto-4-thia-heptanedioic acid, monoethylester,
2,6-dimercapto-4-thia-heptanedioic acid, monopropylester,
2,6-dimercapto-4-thia-heptanediamide,
2,6-dimercapto-4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-4-thia-N,N′-bis(2-hydroxypropyl)-heptanediamide,
2,6-dimercapto-4-thia-heptanedioic acid, dimethylester,
2,6-dimercapto-4-thia-heptanedioic acid, diethylester,
2,6-dimercapto-4-thia-heptanedioic acid, dipropylester,
N-(carboxy-mercapto-methyl)-2-mercapto-malonamic acid,
[(carboxy-mercapto-methyl)-methyl-amino]-mercapto-acetic acid.

19. A dicarboxydithiol according to claim 18, characterized in that it is selected from the group consisting of the following compounds:
2,3-dimercapto-4-methylamino-4-oxo-butanoic acid,
2,3-dimercapto-4-ethylamino-4-oxo-butanoic acid,
2,3-dimercapto-4-propylamino-4-oxo-butanoic acid,
2,3-dimercapto-4-butylamino-4-oxo-butanoic acid,
2,3-dimercapto-4-[(2-hydroxyethyl)amino]-4-oxo-butanoic acid,
2,3-dimercapto-4-[(2-hydroxypropyl)amino]-4-oxo-butanoic acid,
2,3-dimercapto-4-[(2-dimethylaminoethyl)amino]-4-oxo-butanoic acid,
2,3-dimercapto-4-[(3-dimethylaminopropyl)amino]-4-oxo-butanoic acid,
2,3-dimercapto-N,N′-dimethyl-butanediamide,
2,3-dimercapto-N,N′-bis(2-hydroxyethyl)-butanediamide,
2,3-dimercapto-N,N′-bis(2-hydroxypropyl)-butanediamide,
2,4-dimercapto-5-methylamino-5-oxo-pentanoic acid,
2,4-dimercapto-5-ethylamino-5-oxo-pentanoic acid,
2,4-dimercapto-5-propylamino-5-oxo-pentanoic acid,
2,4-dimercapto-5-[(2-hydroxyethyl)amino]-5-oxo-pentanoic acid,
2,4-dimercapto-5-[(2-hydroxypropyl)amino]-5-oxo-pentanoic acid,
2,4-dimercapto-5-[(2-dimethylaminoethyl)amino]-5-oxo-pentanoic acid,
2,4-dimercapto-5-[(3-dimethylaminopropyl)amino]-5-oxo-pentanoic acid,
2,4-dimercapto-N,N′-dimethyl-pentanediamide,
2,4-dimercapto-N,N′-bis(2-hydroxyethyl)-pentanediamide,
2,4-dimercapto-N,N′-bis(2-hydroxypropyl)-pentanediamide,
2,5-dimercapto-6-methylamino-6- oxo-hexanoic acid,
2,5-dimercapto-6-ethylamino-6- oxo-hexanoic acid,
2,5-dimercapto-6-[(2-hydroxyethyl)amino]-6- oxo-hexanoic acid,
2,5-dimercapto-6-[(2-hydroxypropyl)amino]-6- oxo-hexanoic acid,
2,5-dimercapto-6-[(2-dimethylaminoethyl)amino]-6- oxo-hexanoic acid,
2,5-dimercapto-6-[(3-dimethylaminopropyl)amino]-6- oxo-hexanoic acid,
2,5-dimercapto-hexanediamide,
2,5-dimercapto-N,N′-dimethyl-hexanediamide,
2,5-dimercapto-N,N′-diethyl-hexanediamide,
2,5-dimercapto-N,N′-bis(2-hydroxyethyl)-hexanediamide,
2,5-dimercapto-N,N′-bis(2-hydroxypropyl)-hexanediamide,
2,6-dimercapto-7-methylamino-7-oxo-heptanedioic acid,
2,6′ dimercapto-7-ethylamino-7-oxo-heptanedioic acid,
2,6-dimercapto-7-[(2-hydroxyethyl)amino]-7-oxo-heptanedioic acid,
2,6-dimercapto-7-[(2-hydroxypropyl)amino]-7-oxo-heptanedioic acid,
2,6-dimercapto-heptanediamide,
2,6-dimercapto-N,N′-dimethyl-heptanediamide,
2,6-dimercapto-N,N′-bis(2-hydroxyethyl)-heptanediamide,
2,6-dimercapto-N,N′-bis(2-hydroxypropyl)-heptanediamide,
l'acide 2,7-dimercapto-8-methylamino-8-oxo-octanedioic acid,
l'acide 2,7-dimercapto-8-ethylamino-8-oxo-octanedioic acid,
2,7-dimercapto-8-[(2-hydroxyethyl)amino]-8-oxo- octanedioic acid,
2,7-dimercapto-8-[(2-hydroxypropyl)amino]-8-oxo-octanedioic acid,
2,8-dimercapto-heptanediamide,
2,8-dimercapto-N,N′-dimethyl-heptanediamide,
2,8-dimercapto-N,N′-bis(2-hydroxyethyl)-heptanediamide,
2,8-dimercapto-N,N′-bis(2-hydroxypropyl)-heptanediamide,
2,4-dimercapto-3-oxa-5-methylamino-5-oxo-pentanoic acid,
2,4-dimercapto-3-oxa-5-ethylamino-5-oxo-pentanoic acid,
2,4-dimercapto-3-oxa-5-[(2-hydroxyethyl)amino]-5-oxo-pentanoic acid,
2,4-dimercapto-3-oxa-5-[(2-hydroxypropyl)amino]-5-oxo-pentanoic acid,
2,4-dimercapto-3-oxa-5-[(2-dimethylaminoethyl)amino]-5-oxo-pentanoic acid,
2,4-dimercapto-3-oxa-5-[(3-dimethylaminopropyl)amino]-5-oxo-pentanoic acid,
2,4-dimercapto-3-oxa-pentanediamide,
2,4-dimercapto-3-oxa-N,N′-dimethyl-pentanediamide,
2,4-dimercapto-3-oxa-N,N′-bis(2-hydroxyethyl)-pentanediamide,
2,4-dimercapto-3-oxa-N,N′-bis(2-hydroxypropyl)-pentanediamide,
2,6-dimercapto-4-thia-7-methylamino-7-oxo-heptanedioic acid,
2,6-dimercapto-4-thia-7-ethylamino-7-oxo-heptanedioic acid,
2,6-dimercapto-4-thia-7-[(2-hydroxyethyl)amino]-7-oxo-heptanedioic acid,
2,6-dimercapto-4-thia-7-[(2-hydroxypropyl)amino]-7-oxo-heptanedioic acid,
2,6-dimercapto-4-thia-heptanediamide,
2,6-dimercapto-4-thia-N,N′-dimethyl-heptanediamide,
2,6-dimercapto-4-thia-N,N′-diethyl-heptanediamide,
2,6-dimercapto-4-thia-N,N′-bis(2-hydroxyethyl)-heptanediamide,
2,6-dimercapto-4-thia-N,N′-bis(2-hydroxypropyl)-heptanediamide.
